(19) United States
(12) Patent Application Publication
(10) Pub. No.: US 2007/0197973 A1
(43) Pub. Date: Aug. 23, 2007

(54) APPARATUS AND METHOD FOR DRAWING SAMPLES OF BLOOD

(76) Inventors: Stephen Miko, Roanoke, VA (US); Hunt H. Keith, Newport, VA (US)

Correspondence Address: LATIMER IP LAW, LLP, 13873 PARK CENTER ROAD, SUITE 122, HERNDON, VA, 20171, US

(21) Appl. No.: 11/677,396

(22) Filed: Feb. 21, 2007

Related U.S. Application Data
(60) Provisional application No. 60/774,649, filed on Feb. 21, 2006.

(51) Int. Cl. A61M 5/32

(52) U.S. Cl. 604/174

(57) ABSTRACT

The present invention provides a device for drawing blood from a patient, and methods of drawing blood from a patient using the device. The device comprises holders for collection vials and holders for a luer assembly, and does not require mechanical movement of any part of the device. The device is easy to fabricate, simple to use, and long-lasting.

### CROSS-REFERENCE TO RELATED APPLICATIONS

This application relies on the disclosure of and claims the benefit of the filing date of U.S. provisional patent application No. 60/774,649, filed 21 Feb. 2006, the entire disclosure of which is hereby incorporated herein in its entirety by reference.

### BACKGROUND OF THE INVENTION

1. Field of the Invention

The present invention relates to the field of health care. More specifically, the present invention relates to the process of drawing blood from a patient for clinical analysis, and to devices for use in that process.

2. Description of Related Art

The past thirty years have seen a tremendous increase in the amount of information that can be obtained from blood and blood products. Most importantly among that information is information on the health, both general and specific, of the patient from whom the blood has been taken. For example, a sample of blood may now be analyzed for blood sugar content, which is indicative of the general nutritional state of the patient and indicative of the specific status of the individual with respect to various diseases and disorders of blood glucose homeostasis, such as diabetes. Likewise, a sample of blood may be analyzed for the levels of various salts and minerals, which can give medical practitioners clues as to the functioning of various organs and tissues within the patient, including the liver and kidneys. In addition, blood samples are routinely tested for biological products, such as proteins, hormones, and metabolic products, to give an indication of the functional state of various organs and tissues (e.g., adrenal glands, hypothalamus). In addition, blood is now routinely assayed to determine numbers and ratios of blood cells per unit volume, which can give an indication of the immune state of the patient and the general health of the patient. Furthermore, samples of blood are now routinely assayed for the presence of any number of infectious agents, such as bacteria and viruses (e.g., human immunodeficiency virus, hepatitis C virus). The ability to screen relatively small samples of blood (e.g., 10 ml.) from patients for a vast number of characteristics has had a tremendous positive impact on the quality of health services provided to patients by medical practitioners, and the quality of service will continue to rise as more new, faster, and more accurate assays are developed by scientists.

In order to obtain sufficient quantities of blood for the various tests now routinely performed, or which are to be performed on a special basis based on one or more clinical symptoms of a particular patient, medical providers often must take multiple vials of blood from a patient in a single sitting. To do so, the medical provider, typically a nurse, must insert a needle into a suitable vein of the patient and fill the appropriate number of collection vials with blood. This is now typically achieved by way of a single needle prick into a vein of the patient, where the needle is attached to a flexible transfer tube that comprises a needle on the opposite end, which is inserted into a collection vial. When one vial is filled to the appropriate level, the medical provider stops the flow of blood through the transfer tube, switches the needle from the filled vial to an empty vial, and allows the blood to flow through the transfer tube once again, filling the second vial. This procedure is repeated as many times as necessary to obtain the needed sample vials of blood. Once all vials are filled, the medical provider removes the needle from the patient's vein and discards the needle/tube/needle assembly.

In the process of drawing blood, the medical provider must stabilize the needle in the patient's arm while inserting and removing the needle on the other end of the transfer tube into and out of the various blood collection vials. Such a procedure has been found to be quite difficult, and can require the assistance of a second provider to ensure that the needle in the patient's arm is not dislodged, and that transfer of the other needle from one collection vial to another occurs sufficiently rapidly that no, or very little, blood is lost, and that the blood does not clot in the tube. An early solution to this difficulty was to provide a luer assembly for the needle that is used to insert into the blood collection vials. In essence, the luer assembly provides a holder for the needle, and a guide tube for inserting the collection vials. The needle is arranged in the luer assembly such that it punctures a resealable closure at the end of the collection vial when the collection vial is inserted into the luer assembly tube. Upon removal of the collection vial, the needle remains in the luer assembly, ready for use on a subsequent collection vial, which may be inserted into the luer assembly tube.

While the use of such a luer assembly has proven to be an advantageous solution to problems associated with blood drawing, the process is still cumbersome and often difficult. For example, the medical provider must still maintain the needle in the proper position in the patient's arm while attempting to insert and remove each collection vial into the luer assembly. The medical provider must also attempt to keep all of the various collection vials in close proximity to each other and to the luer assembly to enable rapid transfer of filled collection vials and empty collection vials. Keeping all of the various vials in order, making efficient transfers of vials, and maintaining the needle in the patient's arm properly can be a difficult task, particularly when the patient is uncooperative or otherwise unable to control his body.

To improve the blood draw process, various devices have been developed over the years. For example, U.S. Pat. No. 4,951,685 to Blair discloses a blood drawing system comprising a sliding-controlled tubular cartridge for holding collection vials. In practice, the user slides the cartridge forward to engage the collection vial with a needle assembly, which is ultimately connected to a needle inserted into a patient's arm. Engagement of the collection vial with the needle assembly permits flow of blood into the collection vial. Upon filling of the vial, the user slides the cartridge back away from the needle and removes the vial. A new vial may then be placed in the cartridge, and the process repeated.

In addition, the “Tube Jockey” product line from MarketLab (researchML.com) provides a 4-place tube holder for blood draw vials, which can optionally be coupled to a hook-and-loop band for securing to a phlebotomist's arm. The products are disclosed as being suitable for holding 13-16 mm blood draw vials, and for conveniently holding the vials during blood drawing procedures.

Further, U.S. Pat. No. 6,126,609 to Keith et al. discloses an apparatus for taking blood samples from a patient. The apparatus comprises a automatic sliding mechanism for moving a blood collection vial into contact with a luer assembly, which is secured to a base plate. In essence, the automated sliding mechanism grasps a collection vial and slides it forward to engage the collection vial with the luer assembly, which is ultimately connected to a needle inserted into a patient's arm. As with the process of U.S. Pat. No. 4,951,685, engagement of the collection vial with the needle assembly permits flow of blood into the collection vial. Upon filling of the vial, the automated system slides the cartridge back away from the needle and removes the vial. The automated system may then grasp a new vial, place it in position for engagement with the luer assembly, engage the assembly, and fill the collection vial. This process may be repeated.

Although numerous advances have been made in the art, there still exists a need for new devices to aid phlebotomists, nurses, and other medical practitioners in drawing blood from patients. Indeed, while various devices are known in the art, additional attention needs to be paid to designs that provide patient comfort and safety and improved convenience and safety for the medical practitioner drawing the blood.

### SUMMARY OF THE INVENTION

The present invention provides an improved device or apparatus for drawing blood from a patient. The device is relatively simple in design and construction, yet superior to other such devices known in the art in many respects. For example, it is lightweight, streamlined, sturdy, and versatile in its various configurations. It is also relatively inexpensive to fabricate and adaptable. In addition, it provides a convenient and organized means for placement and retention of most or all of the supplies necessary for drawing of blood from a patient, including, but not limited to, secure placement of a luer assembly and secure placement of blood collection vials. It has no mechanical moving parts; therefore, mechanical failures and costly repairs are minimized. The design of the device permits users to know exactly where some or all of their supplies (e.g., vials) are located, allowing quick access to them an minimizing the chance of vials rolling to inaccessible places or falling from trays and shattering. The design also provides support for luers and luer assemblies, allowing quicker and safer inserting and withdrawing of vials. In addition, the device allows for a more streamlined blood drawing process, which results in less chance of needle injury to patients and health care providers. Furthermore, elements of the device can be removably fastened to the device to facilitate removal and replacement if an element becomes worn or broken. Conversely, the present invention relies on the interaction of the practitioner to handle collection vials, which can be an advantage over certain automated systems in that it provides flexibility in implementation by allowing the user to make decisions at the actual point of care as to which vials to fill and in which order, and permits labeling of vials, if desired.

In a first aspect, the invention provides a device or apparatus (used interchangeably herein) for drawing blood from a patient. In general, the device comprises a base to which is secured: 1) one or more holders for one or more blood collection vials. In certain embodiments, the device further comprises one or more holders for one or more luer assemblies. The device, and in particular the base, does not, however, include any mechanical or automated elements that provide mechanical movement for a collection vial, a luer assembly, or any other supply or reagent used in the process of drawing blood from a patient.

In another aspect, the invention provides a method of drawing blood from a patient. In general, the method comprises using the device of the invention to collect one or more samples of blood from a patient. Typically, the method comprises securing one or more blood collection vials to the device, drawing blood from a patient, and collecting the blood using the device of the invention. In embodiments, the method further comprises securing one or more luer assemblies to the device. Due to the stability and portability of the device of the present invention, the method of the invention can be practiced in parts at different times and by different practitioners. That is, some of the method steps may be performed by one person in one location, while other steps may be performed by another person and/or at another location. For example, in practice of the method of the invention, securing of the luer assembly to the base can be accomplished at a nurse's station at the start of a work shift by a nurse's aid, whereas securing of the collection vials and drawing of the patient's blood may be performed by a nurse at a later time.

### BRIEF DESCRIPTION OF THE DRAWINGS

The accompanying drawings, which are incorporated in and constitute a part of this specification, illustrate several embodiments of the invention, and together with the written description, serve to explain certain principles of the invention.

FIG. 1 is a perspective view of one embodiment of the device of the invention, in which multiple blood collection vial holders are located on the base in conjunction with a luer assembly holder.

FIG. 2 is a perspective view of one embodiment of the invention, in which a luer assembly holder is aligned on the base parallel to multiple blood collection vial holders.

FIG. 3 is a perspective view of one embodiment of the device of the invention, showing that various size collection vials can be accommodated on the device and in a configuration in which all of the vials are located on one side of the luer assembly.

FIG. 4 is a perspective view of one embodiment of the device of the invention, which includes a strap for securing the device to an object, such as a phlebotomist's wrist.

FIG. 5A is a side view of one embodiment of the device of the invention, depicting a configuration of the device comprising channels on the underside of the base for securing the device to an object, such as an arm of a chair.

FIG. 5B is a bottom view of the embodiment depicted in FIG. 4A, showing the placement of the channels.

### DETAILED DESCRIPTION OF VARIOUS EMBODIMENTS OF THE INVENTION

Reference will now be made in detail to various exemplary embodiments of the invention, an examples of which are illustrated in the accompanying drawings. It is to be understood that the following detailed description is provided to describe in detail various embodiments of the invention, and is not intended as a limitation of the invention to any one particular embodiment or any particular combination of elements described in two or more specific embodiments.

In one aspect, the invention provides a device for drawing blood from a patient. In general, the device comprises a base, at least one collection vial holder. In embodiments, the device further comprises at least one luer assembly holder. The device base comprises a top surface, a bottom surface, and four side surfaces. The collection vial holder is connected to the top surface of the device base. Likewise, when present, the luer assembly holder is connected to the top surface of the device base. As a general matter, the device does not comprise moving parts, particularly on or directly connected to the top surface or one or more side surfaces of the device.

The collection vial holder(s) and, when present, the luer assembly holder(s), are connected to the base through any conventional means. For example, they may fastened or attached to the device base with a mechanical fastener, such as, but not limited to, a screw, staple, bolt and nut combination, rivet, nail, tack, etc. Likewise, they may be fastened or attached to the device base with a chemical fastener, such as an adhesive, through welding or fusion of a holder to the device base, and the like. In addition, they may be fastened or attached to the device base through use of one or more other physical properties of the device base, the holder, or both, such as through friction fit of the two. Each of the holders may be attached or connected to the device base independently, using a fastener that may be the same or different than one, some, or all of the other fasteners used to fasten other holders to the device base.

Thus, the invention provides a device comprising a base and at least one means for holding at least one luer assembly and at least one means for holding at least one collection vial. The collection vial holding means and luer assembly holding means are connected to the base by any suitable fastening means, including, but not limited to permanent fastening means and removable or detachable fastening means. For example, the holding means may be attached to the base by way of screws, bolts, rivets, staples, nails, tacks, or other mechanical fastening means. Alternatively, the holding means may be attached to the base by way of adhesive, chemical fusing of the holding means and base, welding or other like means. Alternatively, the holding means may be connected to the base by way of a friction fit between a portion of the holding means (e.g., a bottom plate) and the base of the device. For example, a recess may be present in the device base that is engineered and designed to accept a base of the holding means in such a way that the holding means is held tight to the device base by way of friction between the holding means base and the recess in the device base. The holding means may thus be snapped into place into the device base, then optionally removed at a later date if desired, for example for replacement or cleaning. Those of skill in the art can immediately envision other suitable means for connecting the holding means to the base, and any such means may be used in accordance with the present invention.

The device base may be fabricated in any suitable size and shape. That is, those of skill in the art can immediately envision numerous sizes and shapes that can be advantageously used in practice of the method of the invention, as described herein, and can fabricate the device base to accommodate one or more of these advantages, such as the ability to hold various numbers and sizes of collection vials. In general, the device base is large enough to hold at least one collection vial for blood or other biological fluids. In embodiments, it is large enough to also hold at least one luer assembly. Typically, collection vials for biological fluids, such as blood, are about 5 cm to 10 cm in length, and about 1 cm to 2 cm in diameter. Furthermore, typically, luer assemblies for use in medical procedures are about 5 cm to about 10 cm in length, and about 1 cm to 3 cm in diameter. Thus, in general, the device base can range from about 10 cm by 10 cm (about 4 inches by about 4 inches) to about 25 cm by about 25 cm (about 10 inches by about 10 inches). Various particular embodiments comprise a base that is about 20 cm by about 20 cm (8 inches by 8 inches), about 20 cm by about 25 cm (8 inches by 10 inches), about 10 cm by about 20 cm (4 inches by 8 inches), and about 10 cm by about 15 cm (4 inches by 6 inches). The device base may also be designed and fabricated in numerous shapes, and is thus not limited to a square design. For example, it may be fabricated in the shape of a rectangle or polygon (e.g., pentagon, hexagon, octagon), or a rounded shape, such as a circle or oval. Exemplary embodiments of circular shapes comprise a diameter of about 15 cm (6 inches), about 20 cm (8 inches), and about 25 cm (10 inches). Of course, these ranges and shapes are provided simply to describe typical sizes and shapes; those of skill in the art are free to select other sizes and shapes that are outside of these general ranges and shapes while still achieving a device according to the present invention.

The device base may be fabricated out of any suitable material. Preferably, the base is fabricated out of a material that is sturdy and resilient to physical and chemical damage. Preferably, it is made of a material that may be cleaned and/or disinfected with typical cleaners and/or disinfectants use in medical settings. For example, it is preferable that the device base be fabricated out of a material that may be repeatedly cleaned with aqueous cleaners, such as soap and water. It is also preferable that it be resistant to degradation by at least some organic solvents and cleaners, such as those comprising alcohols (e.g., ethanol, isopropanol). Numerous such materials are known and widely used in the medical device and supply arts, and any such material may be used in accordance with the present invention. For example, the device base may be fabricated from glass, a polymeric plastic material, or metal. It may also be made of a paper product, such as cardboard that preferably has been reinforced by coating or impregnating it with a polymeric material. Among the preferred metals are aluminum and stainless steel. Among the preferred polymeric materials and/or plastics are thermosets, elastomers, and thermoplastics, such as polycarbonates, polyethylenes, polypropylenes, polyvinyl chlorides, and polystyrenes.

As a general matter, the device base comprises a top surface, a bottom surface, and at least one side surface. The number of side surfaces will be a function of the overall shape of the device (e.g., a circular device will have one side surface, a square or rectangular device will have four side surfaces, etc.). Although the overall design of the device base is not critical to the invention, and those of skill in the art are capable of developing numerous designs, as a practical matter, the relative proportions of the top, bottom, and side surfaces are preferably set such that the device is conveniently portable while accommodating the desired number of luer assemblies and collection vials. Non-limiting examples of suitable ranges for sizes of the top and bottom surfaces are provided above. Non-limiting examples of ranges for sizes of the side surfaces include any size between about 0.1 cm to 5 cm. Preferably, the side surface has a size of between about 0.5 cm to about 2 cm, such as about 1 cm. Of course, when relatively strong materials are used to fabricate the base (e.g., stainless steel), the thickness of the device base may be relatively small, whereas when relatively weak materials are used (e.g., a paper product, such as cardboard).

The top surface of the device base connects to holders for at least one collection vial. In embodiments, it also connects to holders for at least one luer assembly. The holders for each of these medical supplies can be any suitable size or shape, and may be made from any suitable material. The sizes and materials for each holder may be selected independently of all others. For example, the holders may be fabricated from flexible or semi-flexible materials, such as any of a number of plastics and metals. In preferred embodiments, the holders are fabricated as friction-fit C clamps that expand during placement of a vial or luer assembly in the holder, then contract again upon complete insertion of the vial or assembly into the clamp. Such friction fit C clamps are well known to those of skill in the art, and are widely available from plastics and metal fabricators. In other embodiments, the holders are strips or pads (in any suitable shape) of one member of a hook-and-loop fastener pair. In these embodiments, the complementary pair member (e.g., the hook member, where a loop member is connected to the device base) is connected to the collection vial or luer assembly, to provide a complete hook-and-loop pair when the vial/luer is connected to the device base. Other embodiments include holders that have more than two functional parts, which taken together form a single holder. In general, the holders are attached to the device base by any suitable fastening means, as discussed above.

The bottom surface of the device, like the top surface, is typically flat. It is also typically of the same approximate size and shape as the top surface. It may be a smooth surface or may be rough to improve friction and thus reduce movement of the device when placed on a surface, particularly a smooth surface.

In embodiments one or both of the bottom surface and top surface comprise a rough texture to increase friction and thus the “holding power” of the device for objects that are placed on the surface, but not securely attached. In embodiments, the bottom surface comprises a rough texture while the top surface does not. The rough texture may be imparted to the surface(s) at the time of fabrication of the surface(s) or at a later time by modifying the surface, per se, or by adding a top coating (e.g., and adhesive-backed layer) onto the surface.

The device base may be a unitary element, fabricated from the same material throughout. Alternatively, among other things, it may be fabricated from two or more substances. For example, it may be fabricated from a wood core coated by two plastic surfaces. It thus may be a solid element or a laminated element. Design of the overall 3-dimensional shape of the base is an option left to the choice of the practitioner. It is envisioned that numerous 3-dimensional shapes may be fabricated, including, but not limited to, those that have a flat top surface and a bottom surface that is concavely curved to accommodate a human arm; and those that have a flat top surface and a bottom surface that contains a recess running from one end to another to accommodate the arm rest of a chair or bed.

In an exemplary embodiment, the bottom surface of the device base is modified to include one or more projections. These projections function to aid in stabilizing the device on various objects. For example, the base may be modified to include projections that form one or more channels for receiving one or more parts or surfaces of a piece of furniture, piece of hospital equipment, or body part. In exemplary embodiments, a channel is formed lengthwise or width-wise across the bottom surface of the device base to permit the base to be stabilized when placed on a larger object. In preferred embodiments, the projections forming the channel are moveable (and further preferably having the ability to be maintained or “locked” in a particular position), such that the width of the channel may be adjusted to accommodate multiple different sizes of objects.

In other exemplary embodiments, one or more modifications are made to the base to allow for one or more fasteners to be connected. The fasteners may be designed to fasten the device to any object, including but not limited to, a body part such as an arm, a portion of a piece of furniture, and a portion of a piece of hospital equipment (e.g., a hospital bed). In one particular exemplary embodiment, the fastener is a strap designed to removably affix the device to a health care professional's arm, for example at the writs or forearm.

Of course, similar modifications as those described above with respect to the bottom surface of the base, may be made to one or more side surfaces and the top surface, where appropriate. That is, the device base may be modified at any number of places to include the features described above without departing from the invention, as envisioned.

In addition, the device base may comprise other elements, such as holders for one or more additional pieces of medical equipment. For example, the top surface of the device base may include clamps for holding sterile or used needles, tissues, gauze, or other materials for wiping or drying surfaces, sterilizes (such as alcohol, iodine, and the like), fresh or used gloves or other medical garments, and trash receptacles (e.g., biohazard bags). Of course, the device base bottom surface or one or more side surfaces may comprise one or more holders instead of or in addition to the holders on the top surface. In embodiments, the holder is a removable adhesive strip, which may or may not also serve as a closure for a bag or container. In embodiments, the additional holder is designed to hold a writing instrument, such as a ball point pen or ink marker (e.g., a marker for labeling vials).

In certain embodiments, the device of the invention is provided as a combination of base and one or more holders for a collection vial and, optionally, one or more holders for a luer assembly. In other embodiments the device further comprises one or more luer assemblies (or portions thereof) removably attached to the base. For example, in embodiments, the device comprises a base and one or more holders for collection vials. In other exemplary embodiments, the device comprises a base and a holder for a luer assembly, a luer assembly in contact and removably held by the luer assembly holder, and two or more holders for collection vials. In yet other exemplary embodiments, the device comprises a base, a holder for a luer assembly, a luer assembly in contact and removably held by the luer assembly holder, and two or more holders for collection vials, each of which in contact with and removably holding a collection vial.

One basis for the invention is the simplicity of design and ease of use of the device of the invention. To provide additional ease of use and versatility, the device may be configured in numerous different ways. That is, the number of holders for each of the collection vials and optional luer assemblies may be varied according to need or desire. Likewise, the orientation of the vial holders, and/or the orientation of the vial holder(s) and the luer assembly holder(s) may be varied with respect to the directionality of the base and with respect to the orientation of the other holders. Thus, in embodiments, all holders are aligned such that all vials and luer assemblies, when properly held in position by the holders, are parallel or substantially parallel with each other. Alternatively, in embodiments, the luer assembly holder is oriented such that it is at a 90° angle with respect to the vial holders. Then again, in embodiments, some of the vial holders can be aligned with each other, while other vial holders are aligned at a 90° angle with regard to those holders. Furthermore, in embodiments all holders are positioned on one side of the top surface of the base, providing a convenient and (where a padded rest is provided) comfortable area for a patient to rest a body part, such as a hand or arm. Those of skill in the art are free to elect from among the numerous permutations of alignment schemes to suit a particular desire. In embodiments where a “butterfly” needle is not used, such as when a luer assembly comprising a needled directly attached to the luer and where the luer assembly is positioned adjacent to or touching the patient's skin while the needle is inserted into the patient' blood vessel, the device typically does not comprise a luer assembly holder, although it may be present for storage of the luer assembly after use.

Variations in configuration are not limited to the angles at which holders are aligned. Indeed, numerous other configuration schemes can be accommodated in the present device. For example, the placement of holders with respect to each other on the device base can be varied. Thus, in some embodiments, the luer assembly holder may be positioned in the middle of the device base, and an equal number of vial holders positioned on each side of the luer assembly holder. Alternatively, the luer assembly holder may be positioned at one end or near one edge of the base, and all of the vial holders positioned adjacent it and toward the center of the base. Then again, for example, a luer assembly holder may be positioned at one end or near one edge of the device base, a second luer assembly holder may be positioned at the opposite end or near the opposite edge (but on the same surface), and one or more vial holders positioned between the two.

Within the various configurations of the device are those configurations that include one or more luer assemblies, one or more collection vials, or one or more of both. The luer assembly may be any of the numerous luer assemblies known in the art and commercially available. Likewise, the collection vial may be any of the numerous vials known in the art and commercially available. Those of skill in the medical art are well aware of the various types, sizes, materials of fabrication, and shapes of luer assemblies and collection vials, and may select appropriate ones based on any number of parameters, and such a selection is well within the skill level of those of skill in the art.

In an additional, the invention provides a method of drawing a fluid from a patient. Although not so limited, for the remainder of this disclosure, drawing of blood is exemplified. In essence, the method comprises using the device of the invention to collect one or more samples of blood from a patient. As a general matter, the method comprises inserting a needle into a blood vessel of a patient; allowing blood from the patient to flow through the needle into a luer assembly; operationally connecting a collection vial to the luer assembly; permitting the blood to flow through the luer assembly to the collection vial; collecting a desired amount of blood; and removing the collection vial. In embodiments, the luer assembly comprises a tube through which blood flows from the patient's body to the portion of the luer assembly that contacts the collection vial. In embodiments, the method further comprises providing the luer assembly as a component removably fastened to the device of the invention. Typically, but not exclusively, in these embodiments, the luer assembly comprises a “butterfly” needle. In other embodiments, the luer assembly is not provided as part of the device, but is placed in a holder of the device after drawing of the blood is complete. The method can further comprise providing one or more luer assemblies as a removable element of a device of the present invention or, alternatively, providing one or more luer assemblies and removably affixing it or them to a device of the present invention. Likewise, the method may further comprise providing one or more collection vials as a removable element of a device of the present invention or, alternatively, providing one or more collection vials and removably affixing it or them to a device of the present invention.

Typically, the method comprises removably securing, attaching, affixing, etc. one or more luer assemblies and/or one or more blood collection vials to the device, drawing blood from a patient, and collecting the blood using the device of the invention. The method may further comprise removing a collection vial from a holder of the device; placing it in operational contact with a luer assembly; collecting blood from the patient into the collection vial; and replacing the vial in its holder. The method may further comprise removing another vial from its holder; placing that vial in operational contact with a luer assembly; collecting a desired amount of blood; and replacing the collection vial into its holder. This optional further series of actions may be repeated any number of times, and is preferably repeated a sufficient number of times to fill all of the collection vials that the device can accommodate or that the device was provided with.

The method can further comprise attaching the device of the invention to an object. Attachment can be by any suitable means, but is preferably by way of a fastener that is part of the device, such as by way of a channel formed in or at the bottom surface of the device base or by way of a strap that is affixed to the device base.

As used herein, a vial is in operational contact with a luer assembly when the vial is in physical contact with the luer assembly in such a way that blood flowing through the luer assembly can flow, directly or indirectly, into the collection vial without the need to use external forces, and without the loss of substantial amounts of blood. Thus, mere contact of the vial and luer assembly is insufficient to provide operational contact.

Also, as used herein, a patient is any animal. Thus, it can be a human or any other mammal. It thus may be a dog, cat, or other companion animal; or a horse, cow, sheep, or other agricultural animal. The method and device thus have applicability not only to human health and human medical settings, but to animal health and veterinarian settings as well.

Due to the stability and portability of the device of the present invention, the method of the invention can be practiced in parts at different times and by different practitioners. That is, some of the method steps may be performed by one person in one location, while other steps may be performed by another person and/or at another location. For example, in practice of the method of the invention, securing of the luer assembly to the base can be accomplished at a nurse's station at the start of a work shift by a nurse's aid, whereas securing of the collection vials and drawing of the patient's blood may be performed by a nurse, phlebotomist, or lab technician at a later time.

Of course, the order in which the steps are performed is not critical, although in situations where a luer assembly is used that does not permit one to restrict the flow of blood when the luer assembly is not in operational contact with a collection vial, it is preferred that the user manually restrict blood flow before or immediately after insertion of the needle into the blood vessel, or that a collection vial be placed in operational contact with the luer assembly prior to insertion of the needle into the blood vessel.

As can be seen from the above description, practice of the invention relies, at least in part, on manual movement of the device, luer assembly, and/or collection vials to effect collection of blood. While not an automated system, manual interplay within the method has advantages, such as adaptability to rapid changes in the immediate environment about the collection site, ability to monitor and adjust blood flow from the patient into each vial individually, and the like.

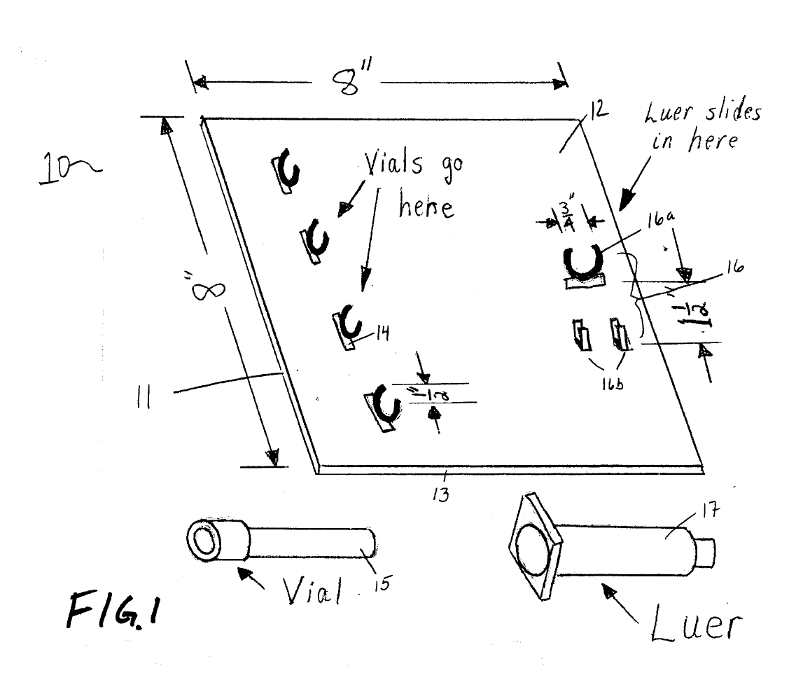

Turning now to the Figures, which are intended to be purely exemplary of the invention, and should not be considered as limiting the invention in any way, FIG. 1 depicts one embodiment of the device of the invention. In this embodiment, a device 10 of the invention is provided. The device 10 comprises a plastic (e.g., plexiglass) base 11, which is in the shape of a square measuring 8 inches (about 20 cm) per side. The base 11 comprises a top surface 12, four side surfaces 13, and a bottom surface (not depicted). The top surface 12 comprises four holders 14 for collection vials 15, which are provided as unitary elements comprising flexible plastic C-clamps for removably attaching collection vials to the device 10. The four holders 14 are permanently affixed to the base 11 by way of chemical fusing. The top surface 12 of the base 11 further comprises a holder 16 for a luer assembly (which comprises a luer 17 and a needle/tube element, which is not depicted). The luer holder 16 is a three-part element comprising a plastic C-clamp 16a for reversibly holding the barrel of a luer 17, and two luer plate holders 16b, for holding and stabilizing the luer 17 when being held in the holder 16. Holder 16 is permanently affixed to the top surface 12 of base 11 by way of chemical fusing.

In practicing the invention with the device depicted in FIG. 1, one to four collection vials 15 are clamped into holders 14, and a luer 17 is clamped into luer holder 16. A luer assembly is fashioned by inserting a flexible tube comprising a needle for inserting into a patient's vein (e.g., a butterfly needle) and a needle for operational contact between the luer assembly and a collection vial. Upon insertion of the needle into the patient's vein, a collection vial 15 is inserted into the luer 17 of the luer assembly, and operational contact between the luer assembly and the collection vial 15 is achieved. The collection vial 16 is filled to a desired level, and then removed from the luer assembly and placed back into its holder 14. Another vial 15 is then removed from its holder 15 and inserted into luer 17 such that operational contact is made and blood flows into the vial 15. Upon collection of a desired amount of blood, vial 15 is removed and replaced into holder 14. This process is repeated until the desired number of vials 15 are filled and re-secured in holders 14. The needle is then removed from the patient's body and the device, now containing filled collection vials, is taken away for analysis of the samples that were collected.

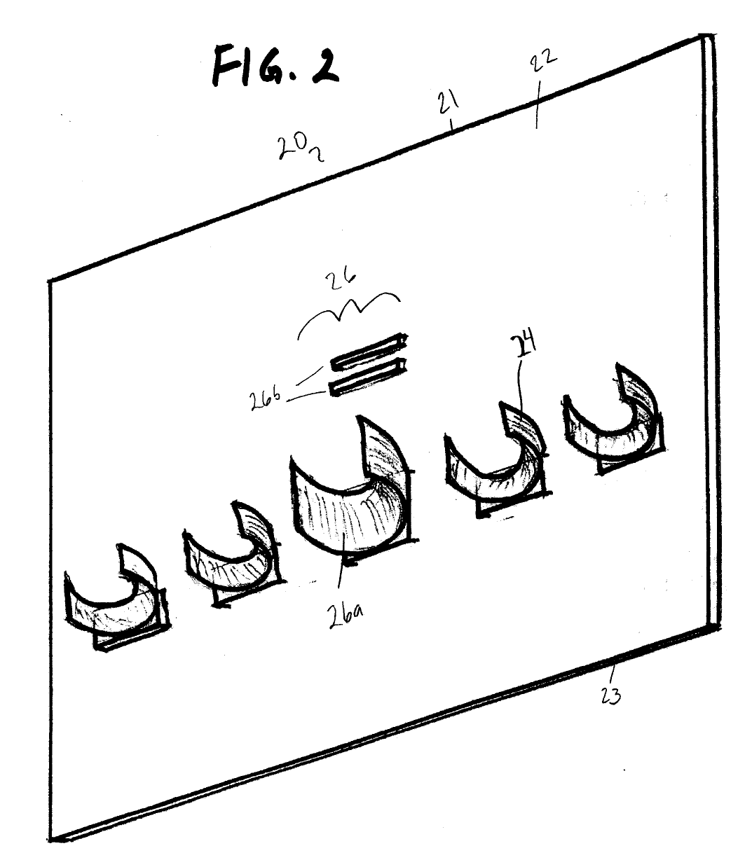

FIG. 2 shows an embodiment of the invention in which a device 20 is provided with four holders for vials and one holder for a luer assembly. The device 20 comprises a base 21 having a top surface 22, four side surfaces 23, and a bottom surface (not depicted). Onto the top surface 22 is permanently affixed four holders 24 for collection vials (not depicted), which are provided as unitary elements comprising flexible plastic C-clamps for removably attaching collection vials to the device 20. The four holders 24 are permanently affixed to the base 21 by way of chemical fusing. The top surface 22 of the base 21 further comprises a holder 26 for a luer assembly (not depicted). The luer holder 26 is a three-part element comprising a plastic C-clamp 26a for reversibly holding the barrel of a luer, and two luer plate holders 26b, for holding and stabilizing the luer when being held in the holder 26. Holder 26 is permanently affixed to the top surface 22 of base 21 by way of chemical fusing. As can be seen, in this Figure, the holder 26 for the luer is located generally in the middle of the base 21, and is flanked on each side by two holders 24 for collection vials, where all of the holders are arranged in parallel with each other. This is in contrast to the configuration depicted in FIG. 1, in which the luer holder 16 is located at one end of the base 11 near an edge, and the four vial holders 14 are arranged near the opposite edge and at a 90° angle with respect to the luer holder 16. In practicing the invention with this exemplary embodiment, the same procedure is employed as described above with regard to the embodiment of the device depicted in FIG. 1.

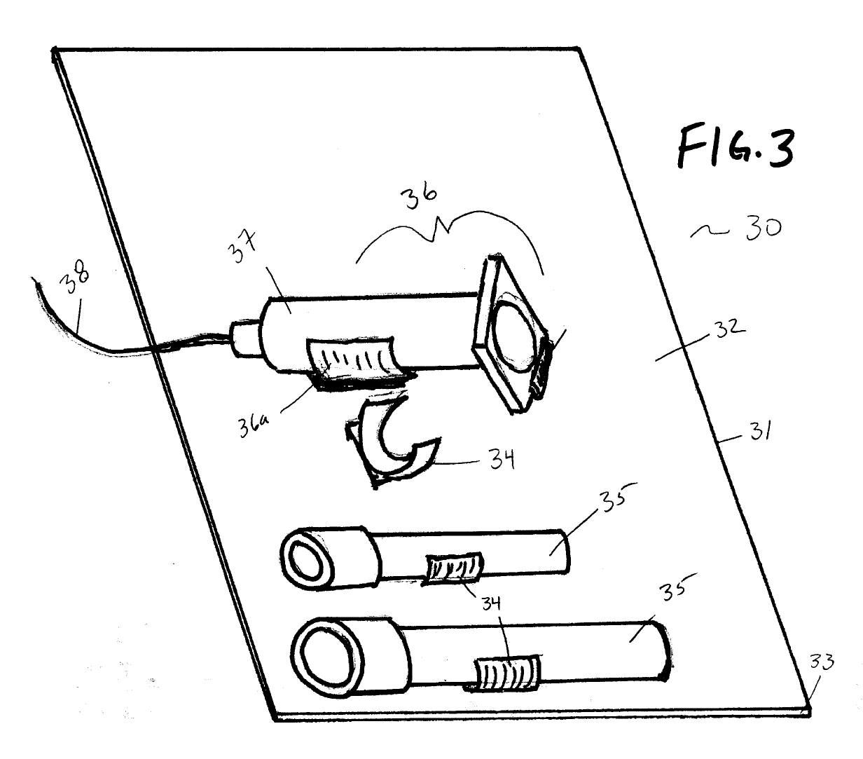

FIG. 3 depicts an embodiment of the device in which device 30 is provided in a configuration that provides an area on top surface 32 for placement of an object, such as the hand or arm of the patient. More specifically, FIG. 3 depicts a device 30 comprising a base 31 having a top surface 32, four side surfaces 33, and a bottom surface (not depicted). The top surface 32 comprises three holders 34 for collection vials 35, which are provided as unitary elements comprising flexible plastic C-clamps for removably attaching collection vials 35 to the device 30. The three holders 34 are permanently affixed to the base 31 by way of chemical fusing. The top surface 32 of the base 31 further comprises a holder 36 for a luer assembly (which comprises a luer 37 and a needle/tube element 38). The luer holder 36 is a three-part element comprising a plastic C-clamp 36a for reversibly holding the barrel of a luer 37, and two luer plate holders 36b (only one is depicted), for holding and stabilizing the luer 37 when being held in the holder 36. Holder 36 is permanently affixed to the top surface 32 of base 31 by way of chemical fusing. Practice of the method of the invention using this configuration of the device of the invention is accomplished as described above.

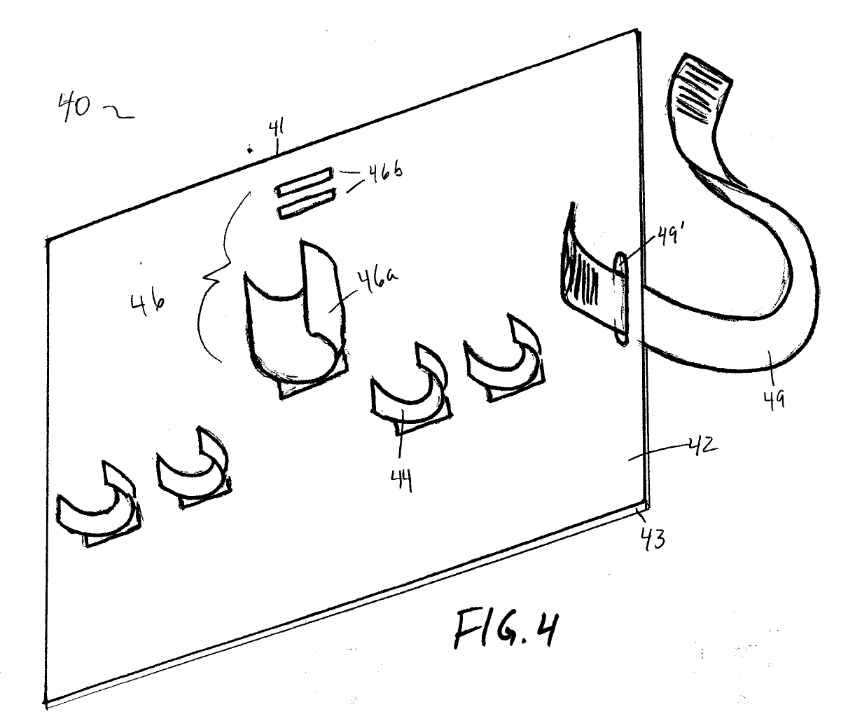

FIG. 4 depicts another configuration of the device of the invention. In this figure, a device 40 is provided with a fastener for fastening the device to an object, such as a patient's arm or leg, or a piece of furniture. Specifically, device 40 comprises a base 41, which comprises a top surface 42, four side surfaces 43, and a bottom surface (not depicted). The top surface 12 comprises four holders 44 for collection vials (not depicted), which are provided as unitary elements comprising flexible plastic C-clamps for removably attaching collection vials to the device 40. The four holders 44 are permanently affixed to the base 41 by way of chemical fusing. The top surface 42 of the base 41 further comprises a holder 46 for a luer assembly. The luer holder 46 is a three-part element comprising a plastic C-clamp 46a for reversibly holding the barrel of a luer, and two luer plate holders 46b, for holding and stabilizing the luer when being held in the holder 46. Holder 46 is permanently affixed to the top surface 42 of base 41 by way of chemical fusing.

Device 40 further comprises strap 49, which is connected to base 41 through an opening 49′ in base 41 that traverses the base 41 from the top surface 42 to the bottom surface. Strap 49 can be used to reversibly affix the device 40 to an object, such as a piece of furniture or a body part of a patient (e.g., leg, arm). In other embodiments (not depicted), base 41 comprises another through-hole or opening at or near the edge of the top and bottom surfaces opposite of opening 49′, through which one end of strap 49 may be passed to aid in securing device 40 to an object.

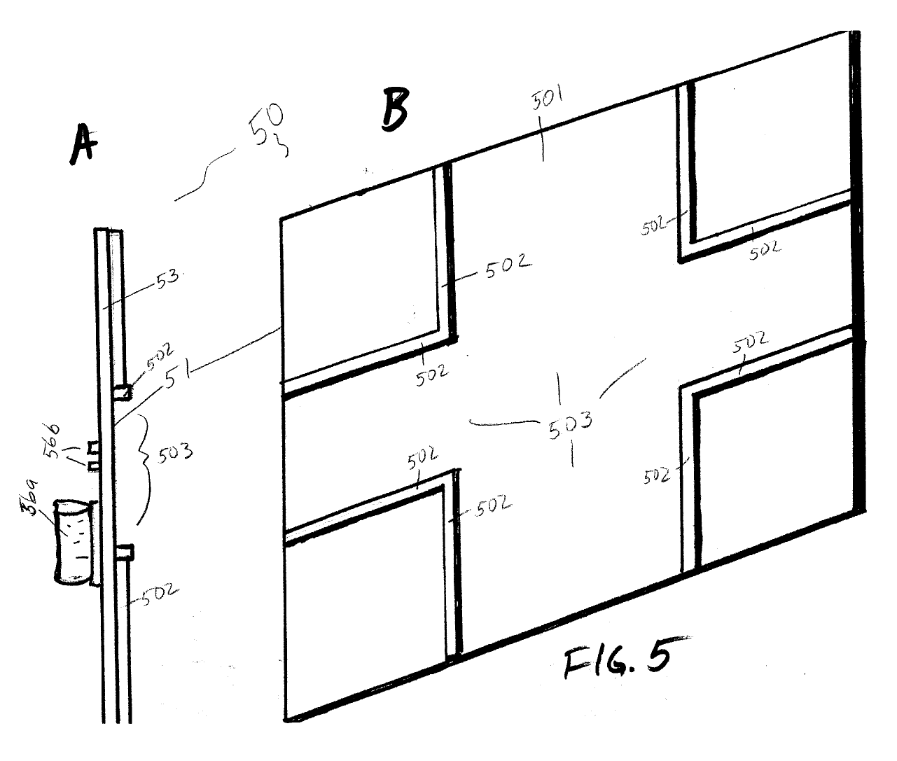

Turning now to FIG. 5, an embodiment of the invention is depicted in which the bottom surface of the device is modified to comprise projections that form channels for securing the device to an object. Specifically, FIG. 5A depicts a side view of device 50, while FIG. 5B depicts a bottom view of the same device. As can be seen, the device 50 comprises a base 51, which comprises a top surface (not depicted), which comprises at least one three-part luer holder 56 (depicted as 56a and two parts 56b). Base 51 further comprises four side surfaces 53 and bottom surface 501. Bottom surface 501 comprises projections 502, which combine with each other to form channels 503 at the bottom surface 501. Channels 503 accommodate objects upon which device 50 rests, such as parts of furniture (e.g., chairs, beds), body parts (e.g., legs), and other solid, sturdy objects that can be used to stabilize device 50 during practice of the method of the invention. In certain embodiments, which are not depicted in this drawing, projections 502 are movable such that the size of channels 503 can be adjusted to accommodate different size objects.

It will be apparent to those skilled in the art that various modifications and variations can be made in the practice of the present invention and in construction of this device without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

1. A device comprising:
a base comprising at least one holder for a vial and at least one holder for a luer assembly, wherein the base does not comprise a movable mechanical element for placing the vial into operational contact with the luer assembly.

2. The device of claim 1, wherein the holder for the vial is designed to reversibly hold the vial in contact with the base of the device.

3. The device of claim 1, wherein the holder for the luer assembly is designed to reversibly hold the vial in contact with the base of the device.

4. The device of claim 1, further comprising a fastener to fasten the device to an object.

5. The device of claim 4, wherein the fastener is a strap.

6. The device of claim 1, wherein the base comprises a bottom face and the bottom face comprises one or more projections that serve to stabilize the device on an object.

7. A device comprising a base comprising
at least one means for holding a collection tube; and
at least one means for holding a luer assembly,
wherein the device does not comprise a movable mechanical element for placing the vial into operational contact with the luer assembly.

8. The device of claim 7, wherein the holder for the vial is designed to reversibly hold the vial in contact with the base of the device.

9. The device of claim 7, wherein the holder for the luer assembly is designed to reversibly hold the vial in contact with the base of the device.

10. The device of claim 7, further comprising a fastener to fasten the device to an object.

11. The device of claim 10, wherein the fastener is a strap.

12. The device of claim 7, wherein the base comprises a bottom face and the bottom face comprises one or more means for stabilizing the device on an object.

13. A method of drawing a fluid from a body, said method comprising
providing a device comprising a base comprising at least one collection vial removably attached to the base, and at least one luer assembly removably attached to the base, wherein the device does not comprise a movable mechanical element for placing the vial into operational contact with the luer assembly;
drawing the fluid from the patient;
depositing the fluid into the collection vial(s) by operationally contacting the vial(s) with the luer assembly or assemblies; and
removing the vial(s) from the luer assembly or assemblies and returning it to its holder,
wherein the contacting of the vial(s) and luer assembly or assemblies does not comprise movement of any mechanical elements of the device.

14. The method of claim 13, wherein the fluid is blood.

15. The method of claim 13, wherein operationally contacting the vial(s) with the luer assembly or assemblies comprises human movement of the vial(s).
